(19) United States
(12) Patent Application Publication
(10) Pub. No.: US 2014/0107222 A1
(43) Pub. Date: Apr. 17, 2014

(54) TREATMENTS FOR SOCIAL LEARNING DISORDERS

(71) Applicant: Indiana University Research and Technology Corporation, Indianapolis, IN (US)

(72) Inventors: Anantha SHEKHAR, Indianapolis, IN (US); D. Wade CLAPP, Indianapolis, IN (US)

(73) Assignee: Indiana University Research and Technology Corporation, Indianapolis, IN (US)

(21) Appl. No.: 14/054,328

(22) Filed: Oct. 15, 2013

Related U.S. Application Data
(60) Provisional application No. 61/714,460, filed on Oct. 16, 2012.

(51) Int. Cl. A61K 31/105

(52) U.S. Cl. CPC A61K 31/105
USPC 514/707

(57) ABSTRACT

The use of Pak1 inhibitors to treat social or learning disabilities is disclosed. In one embodiment patients exhibiting social or learning disabilities as well as abnormally low NF1 activity are administered PAK inhibitors to treat the social or learning disabilities. Reductions in PAK activity have been found to ameliorate the effects of aberrant neurofibromatosis type 1 activity.

### CROSS REFERENCE TO RELATED APPLICATIONS

This application claims priority to U.S. Provisional Application No. 61/714,460, filed Oct. 16, 2012, the contents of which is incorporated by reference in its entirety.

### STATEMENT REGARDING FEDERALLY SPONSORED RESEARCH

This invention was made with government support under grant number RR025761 awarded by the National Institutes of Health. The U.S. Government has certain rights in the invention.

### TECHNICAL FIELD

The present disclosure generally pertains to the fields of neuroscience and psychiatry. More particularly, the present disclosure pertains to methods for treating social learning disorders.

### BACKGROUND AND SUMMARY

Social learning is the product of complex interactions between multiple structures in the CNS, particularly the amygdala and frontal cortex. The prefrontal cortex (PFC) and the basolateral amygdala (BLA) play an important role in regulating social behaviors and learning. The genetics of many of the disorders with disruption of social behaviors such as ASDs, Fragile X and William's syndrome are complex. Also, these disorders have many other complex phenotypic features in addition to social behavior deficits. The genetic and molecular mechanisms involved in the regulation of social learning behaviors have been difficult to decipher.

Disrupted social learning is seen in a wide range of developmental and autism spectrum disorders (ASDs) but little is known about its genetic regulation. Neurofibromatosis type 1 (NF1) is an autosomal dominant disease with mutation in one copy of the NF1 gene (NF1^{+/-}) that affects roughly 1 in 3500 individuals, and frequently presents with learning disabilities, attention deficit hyperactivity disorder, and social deficits similar to ASDs

The NF1 gene encodes neurofibromin, a GAP-like protein that is expressed throughout the central nervous system (CNS). Neurofibromin negatively regulates Ras GTPase activation, thereby reducing the strength and duration of Ras signal transduction. The active form of Ras or Ras-GTP is, in part, responsible for the propagation of the classical Ras-Raf-Mek-Erk (MAPK) and the phosphotidylinositol 3-kinase (PI3K) cascades. Mutation at the NF1 locus increases the output of MAPK and PI3K signal transduction from the cellular membrane to the nucleus resulting in the hyperactivation of Ras and its downstream pathways. P21-activated kinase (PAK1) is a downstream effector regulated by the Rho family of GTPases that mediate diverse cellular functions including cytoskeletal dynamics, vesicular transport, and gene expression. In addition, PAK1 has been shown to positively regulate MAPK activation. Recently, a genetic intercross was developed to disrupt Nf1 and Pak1 expression, and the resulting studies found that the co-deletion of Pak1 (Nf1^{+/-}/Pak1−/−) restored MAPK dependent functions in Nf1 haploinsufficient mast cells (McDaniel et al. (2008).

As reported herein, Nf1^{+/-} mice demonstrate a selective deficit in social learning that is rescued by co-deletion of Pak1 gene. This is quite unlike the recently reported Shank3 mutant mice which prefer interacting with the empty cage and show deficits in overall social behaviors at baseline (Peca et al (2011). The mechanisms underlying the opposing regulation of social learning by Nf1 and Pak1 genes appears to involve disruption of GABA-mediated inhibition and glutamate excitation of the projection neurons of the amygdala and altered expression of important synaptic proteins in the amygdala and PFC. As described herein Pak1 inhibitors are useful for the treatment of NF1-related social learning disorders. Finally, Pak1 inhibitors may also be useful for learning-based remediation of social deficits in some forms of autism spectrum disorders.

### BRIEF DESCRIPTION OF THE DRAWINGS

FIGS. 1a-1k provides data indicating Nf1^{+/-} mice show selective deficits in long-term social learning compared to wild-type (WT, n=12/group) mice. FIGS. 1a-1c depict the time spent sniffing wire cages containing “stimulus” mice in a three-chambered apparatus. No differences were detected between strains in (a) preference for social interaction or (b) short-term social learning (1-3 min following exposure to “test” mouse). c) Unlike WT, Nf1^{+/-} mice show deficits in long-term social learning, as measured by preference for social novelty (24 hrs. following exposure to “test” mouse). No differences were observed in (see FIGS. 1d and 1e) anxiety and avoidance learning (as measured by elevated plus maze), or (f) olfactory habituation (see FIG. 1f). FIGS. 1g-1k show that in the basolateral amygdala (BLA) region Nf1^{+/-} mice have significantly higher presynaptic inhibitory release.

FIG. 2a provides data indicating the co-deletion of Pak1 (Nf1^{+/-}/Pak1^{-/-}) restores MAPK hyperactivation associated with Nf1^{+/-} genotype in neuronal cultures. Western blotting was used to quantify p-ERK1/2 in cultured cortical neurons at basal levels and 2 minutes following stimulation with SCF (10 ng/ml), and total Erk1/2 was used as loading controls. Graphs depict mean±SEM for Nf1^{+/-} (lanes 1 and 2), Nf1^{+/-}/Pak1^{-/-} (lanes 3 and 4), and Pak1^{-/-} (lanes 5 and 6) mice. *P<0.05. FIG. 2b depicts a hypothetical graphical illustration, pathways adapted from Le and Parada (2007) and Cui et al., (2010), showing the interaction of Nf1 and Pak1 gene products in growth factor signal transduction. Neurofibromin, a cytoplasmic GAP-like protein, negatively regulates RAS activation by accelerating the conversion of RAS-GTP to RAS-GDP and increasing RAS-RAF-MEK signal transduction, whereas Pak1 has an activating effect on this pathway.

FIGS. 3a-3i provide data indicating the deficits in long-term social learning and increases in sIPSC and mIPSC frequency seen in Nf1^{+/-} mice can be restored by co-deletion of Pak1. FIGS. 3a-3c depict the time spent sniffing wire cages containing “stimulus” mice in a three-chambered apparatus (refer to insets; Nf1^{+/-}, n=12; Nf1^{+/-}/Pak1^{-/-}, n=11; Pak1−/−, n=6). Similar to applicants' previous findings, no differences were detected between strains in preference for social interaction (FIG. 3a) or short-term social learning (FIG. 3b). Unlike Nf1^{+/-} mice, Nf1^{+/-}/Pak1^{-/-} showed significant preference for social novelty or restored long-term social learning (see FIG. 3c; 24 hrs. following exposure to “test” mouse). No differences were observed in anxiety and avoidance learning (see FIG. 3d or 3e, as measured by elevated plus maze), olfactory habituation or depression-associated behavior (see FIG. 4f). FIGS. 3g-3i indicate that co-deletion of Pak1^{-/-} in Nf1^{+/-} mice normalizes the increases in presynaptic GABA release seen in Nf1^{+/-} BLA projection neurons. FIG. 3(g) provides representative recordings from BLA projection neurons of Nf1^{+/-}, Nf1^{+/-}/Pak1^{+/+} and Pak1^{+/+} mice. FIGS. 3h-3i) provide data regarding the sIPSC and mIPSC frequency for Nf1^{+/-} mice relative to Nf1^{+/-}/Pak1^{-/-} mice. Compared to Nf1^{+/-} mice, Nf1^{+/-}/Pak1^{+/+} exhibited decreased sIPSC and mIPSC frequency, like that of Pak1−/− mice. Nf1+/−, Nf1+/−/Pak1^{-/-}, Pak1^{-/-} showed no differences in sIPSC and mIPSC amplitude. Graphs depict mean±SEM for Nf1^{+/-} (columns 1 and 2), Nf1^{+/-}/Pak1^{-/-} (columns 3 and 4), and Pak1^{-/-} (columns 5 and 6) mice. *P<0.05.

FIGS. 4a & 4b provide data indicating depression-associated behavior of Nf1^{+/-} mice is reduced in Nf1^{+/-}/Pak1^{-/-} mice. FIG. 4a: Nf1^{+/-} and WT mice (n=12/group) show no differences in their percent of time immobile in the forced swim test (FST), a measure of behavioral despair and learned helplessness. FIG. 4b) Nf1^{+/-}/Pak1^{-/-} and Pak1^{-/-} mice show significant decreases in their percent of time immobile in the forced swim test (FST), as compared to Nf1 mice, Swimming “immobility” was assessed during the 2-6 minute interval on day 1 and day 2. Data shown represent the mean±SEM of the percent of time immobile for each group. tP<0.05 for a between-group comparison verses Nf1^{+/-} genotype

FIGS. 5a-5c present the strategy used for determining key proteins involved in the Disruption of social learning by Nf1 haploinsufficiency and its rescue by Pak 1 deletion. FIG. 5a provides protein expression differences in the basolateral amygdala seen in Nf1^{+/-} deficient mice that are rescued by the co-deletion of Pak1^{-/-} gene. Columns represent proteins showing full or partial rescue with Pak1^{-/-} codeletion with Nf1^{+/-}. Column 2 is the locus ID of each protein. Column 3 represents fold changes in protein expression in Nf1^{+/-} mice compared to Wt controls. Column 5 represents definitions for abbreviated proteins in column 1. FIG. 5b is a graph presenting the number of ADAM 22-ir cells in the basolateral amygdala of WT and Nf1+/− mice. Bars represent the mean and error bars represent the standard error of the mean. * indicates a significance with a 2 tailed independent t-test, p=0.027. FIG. 5c contains low (left) and high (right) photomicrographs of ADAM 22-immunoreactive cells in the amygdala of WT (top row) and NF1+/− (bottom row) mice. Central (CeA) and basolateral (BLA) amygdala is indicated with arrows in left row. Dashed lined box in left photomicrographs indicated where cells were counted and also where high magnification photomicrographs to right are taken. Scale bar for photomicrographs in left and right rows and inset are respectively, 25, 75 and 375 μm. This data shows that the protein ADAM 22 is substantially reduced in the brain cells in the amygdala of NF1+/− mice.

### DETAILED DESCRIPTION

### Definitions

In describing and claiming the invention, the following terminology will be used in accordance with the definitions set forth below.

As used herein the term “social learning” relates to knowledge and skills obtained within a social context including for example observational learning, imitation, and modeling, and use of such information to serve as a guide for action on subsequent occasions.

As used herein the general term “PAK”, “PAK polypeptide” or “PAK protein” are equivalent terms used interchangeably to refer to a protein that belongs in the family of p21-activated serine/threonine protein kinases. These include mammalian isoforms of PAK, e.g., the Group I PAK proteins (sometimes referred to as Group A PAK proteins), including Pak1, Pak2, Pak3, as well as the Group II PAK proteins (sometimes referred to as Group B PAK proteins), including Pak4, Pak5, and/or Pak6. Representative examples of PAK include, but are not limited to, human Pak1 (GenBank Accession Number AAA65441), human Pak2 (GenBank Accession Number AAA65442), human Pak3 (GenBank Accession Number AAC36097), human Pak4 (GenBank Accession Numbers NP_{—}005875 and CAA09820), human Pak5 (GenBank Accession Numbers CAC18720 and BAA94194), human Pak6 (GenBank Accession Numbers NP_{—}064553 and AAF82800), human Pak7 (GenBank Accession Number Q9P286).

As used herein, the term “PAK activity,” unless otherwise specified, includes, but is not limited to, at least one of PAK protein-protein interactions, PAK phosphotransferase activity (intermolecular or intermolecular), translocation, etc. of one or more PAK isoforms. Reference to “Pak1 activity” are specifically in reference to Pak1 without regard to the activity of the other PAK isoforms.

As used herein, a “PAK inhibitor” refers to any molecule, compound, or composition that directly or indirectly decreases the PAK activity. In some embodiments, PAK inhibitors inhibit, decrease, and/or abolish the level of a PAK mRNA and/or protein or the half-life of PAK mRNA and/or protein. In some embodiments, a PAK inhibitor is a PAK antagonist that inhibits, decreases, and/or abolishes an activity of PAK. In some embodiments, a PAK inhibitor also disrupts, inhibits, or abolishes the interaction between PAK and its natural binding partners (e.g., a substrate for a PAK kinase, a Rac protein, a cdc42 protein, LIM kinase) or a protein that is a binding partner of PAK in a pathological condition, as measured using standard methods. Thus, binding between PAK and at least one of its natural binding partners is stronger in the absence of the inhibitor than in its presence. Alternatively or additionally, PAK inhibitors may inhibit the phosphotransferase activity of PAK, e.g., by binding directly to the catalytic site or by altering the conformation of PAK such that the catalytic site becomes inaccessible to substrates. In some embodiments, PAK inhibitors inhibit the ability of PAK to phosphorylate at least one of its target substrates, e.g., LIM kinase 1 (LIMK1), myosin light chain kinase (MLCK); or itself, i.e., autophosphorylation.

As used herein a “Pak1 inhibitor” is an inhibitor that reduces the activity of the Pak1 isoform of the p21-activated serine/threonine protein kinases. Similarly a “Pak1 specific inhibitor” is an inhibitor that reduces the activity of only the Pak1 isoform of the p21-activated serine/threonine protein kinases.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes prophylaxis of the specific disorder or condition, or alleviation of the symptoms associated with a specific disorder or condition and/or preventing or eliminating said symptoms.

As used herein an “effective” amount or a “therapeutically effective amount” of an PAK inhibitor refers to a nontoxic but sufficient amount of the inhibitor to provide the desired effect. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term, “parenteral” means not through the alimentary canal but by some other route such as intranasal, inhalation, subcutaneous, intramuscular, intraspinal, or intravenous.

As used herein the term “patient” without further designation is intended to encompass any warm blooded vertebrate domesticated animal (including for example, but not limited to livestock, horses, cats, dogs and other pets) and humans.

As used herein the term “co-administer” in reference to pharmaceutically active agents refers to the administration of two or more active agents wherein the second active agent is administered simultaneously or sequentially to the first active agent, and the first agent retains at least 10% of its activity at the time the second agent is administered.

As used herein a “defective NF1 gene” is a gene that is altered relative to the wild type gene wherein the alteration results in decreased NF1 gene product activity. The decrease in NF1 gene product activity may result from defects in the expression of the gene and/or defects in the encoded protein. For example, the gene may contain one or more mutations that prevents the expression of the gene, or causes reduced expression of the gene, decreases the stability of the mRNA or protein product, or decreases functionality of the produced protein product relative to the native NF1 gene product.

### Embodiments

Much of human behavior is learned or influenced observationally through modeling. More specifically, from observing others one forms an idea of how new behaviors are to be performed, and on later occasions this coded information serves as a guide for action. Just as there are learning disabilities that interfere with academic performance, there are learning disabilities that interfere with acquiring and utilizing social behavior that enable us to function in a society. Children with learning disabilities affecting social skills have difficulty reading the social cues of others. They may not recognize emotional facial expressions or body language that gives clues to guide social behavior. As such a child with this learning disability may not appreciate when they have insulted, upset or frustrated another person. If they cannot recognize the facial expression, then they are at a loss for modulating their own behavior in response. Hence they may carry on with offensive or inappropriate behavior, not recognizing their impact. Further, not only does the child have difficulty reading the social cues of others, the child likely has difficulty viewing his or her own behavior accurately. Hence, they have difficulty gauging their own behavior.

In accordance with one embodiment a method is provided for treating a social learning disability in a patient. The method comprises the steps of identifying a patient with a social learning disability, and administering to said patient a pharmaceutical composition comprising an effective amount of a PAK inhibitor. In one embodiment the method comprises the step of identifying a patient with a social learning disability that also has defective NF1 gene that prevents the expression of the gene, or causes reduced expression of the gene, decreases the stability of the mRNA or protein product, or decreases functionality of the produced protein product relative to the native NF1 gene product. In one embodiment the inhibitor is a mammalian Pak1 inhibitor, more specifically a human Pak 1 inhibitor, and in a further embodiment the inhibitor is specific for human Pak1 activity.

In accordance with one embodiment a patient with a social learning disability is identified based on exhibited behavioral characteristics, including for example, an inability to read the social cues of others, an inability to modulate situationally inappropriate behavior and an inability to respond appropriately to facial expressions or body language of others. In one embodiment the patient to be treated has an attention deficit hyperactivity disorder.

The social learning disability may or may not be accompanied with academic learning difficulties. In one embodiment patients that may benefit from Pak1 inhibitory therapy are identified by personnel in the medical field including for example by a psychologist or psychiatrist. Alternatively, candidates can be screened using standard techniques to identify those individuals that have a defective NF1 gene. Patients exhibiting a social learning disability and having a defective NF1 gene would be targeted for Pak1 inhibitory therapy.

In accordance with one embodiment a patient suffering form a learning or social disability is administered Pak1 inhibitory therapy wherein more than one PAK inhibitor is administered, either simultaneously or sequentially. In one embodiment two or more PAK inhibitors are administered in a single pharmaceutical composition. Alternatively, multiple compositions each comprising one or more PAK inhibitors can be administered to a patient during their therapeutic regiment. In one embodiment where two or more PAK inhibitors are being co-administered, at least one of the PAK inhibitors is a Pak1 inhibitor. In one embodiment two or more Pak1 inhibitors are co-administered, and in one embodiment at least one of the Pak1 inhibitors is a Pak1 specific inhibitor.

Pak1 inhibitors are known to those of skill in the art. Exemplary Pak1 inhibitors include, but are not limited to, staurosporine, 3-hydroxystaurosporine, K252a, CEP-1347, OSU-03012, DW12, FL172 (disclosed in Yi et al., Biochemical Pharmacology, 2010, 80:683-689, the disclosure of which with respect to Pak1 inhibitor compounds is hereby incorporated herein by reference), IPA3 (commercially available from Tocris), PF-3758309, PAK10 (available from Calbiochem), EKB569, TKI258, SU-14813, and other Pak inhibitor compounds as disclosed in U.S. Patent Publication No. 20100317715, paragraphs [0082] to [0121], said paragraphs hereby incorporated herein by reference.

The compositions described herein and their salts may be formulated as pharmaceutical compositions for systemic administration. Such pharmaceutical compositions and processes for making the same are known in the art for both humans and non-human mammals See, e.g., Remington: The Science and practice of pharmacy, (1995) A. Gennaro, et al., eds., 19th ed., Mack Publishing Co. In one embodiment the pharmaceutical compositions comprise aqueous solutions that are sterilized and optionally stored contained within various package containers. In other embodiments the pharmaceutical compositions comprise a lyophilized powder. The pharmaceutical compositions can be further packaged as part of a kit that includes a disposable device for administering the composition to a patient. The containers or kits may be labeled for storage at ambient room temperature or at refrigerated temperature. Additional active ingredients may be included in the composition containing a collagen binding peptide coupled to a nanoparticle, or a salt thereof.

The PAK inhibitory compositions disclosed herein can be formulated using standard techniques for administration to a patient using any standard route of administration, including parenterally, such as intravenously, intraperitoneally, subcutaneously or intramuscularly, intrathecally, transdermally, rectally, orally, nasally or by inhalation. In one embodiment the composition is administered subcutaneously or intramuscularly. In one embodiment, the composition is administered parenterally.

Applicants have demonstrated that defects in NF1 gene leads to deficiencies in learning including for example, deficiencies in social learning. The NF1 gene encodes neurofibromin, which negatively regulates Ras GTPase activation, and thereby reduces the strength and duration of Ras signal transduction. P21-activated kinase (Pak1) is a downstream effector regulated by the Rho family of GTPases that mediate diverse cellular functions including cytoskeletal dynamics, vesicular transport, and gene expression.

Applicants have discovered that the deficit in social learning associated with Nf1+/− mice is rescued by deletion of the Pak1 gene. Accordingly, applicants anticipate that patients having defective NF1 activity can be treated with PAK inhibitors (e.g., a Pak1 inhibitor) to treat learning disabilities and other symptoms or conditions resulting from deficient Nf1 activity. In accordance with one embodiment a method for treating an NF1 deficiency (i.e., decreased NF1 gene expression, decreased NF1 protein product, or decreases functionality of the NF1 protein product relative to the native NF1 gene product) associated learning disability is provided. In one embodiment the method comprises the steps of identifying a patient with defective NF1 activity and administering to said patient a pharmaceutical composition comprising an effective amount of a PAK inhibitor. In one embodiment the composition comprises a Pak1 specific inhibitor. In one embodiment the Pak1 inhibitor is IPA-3. In one embodiment, patients with defective NF1 activity are detected based on an in vitro assay for measuring the binding activity of NF1 recovered from a patient's biological sample. Alternatively, patients that express defective NF1 proteins may be identified based on Western blot analysis of proteins recovered form a biological sample of the patient. In an alternative embodiment patients with defective NF1 activity are identified by analyzing the structure of the patient's NF1 gene to detect mutation that impact NF1 activity. Mutations that impact the expression of the NF1 gene or the activity of the expressed protein include for example, frameshift, nonsense, missense, splicing alteration and deletion mutations. Detections of these mutations can be accomplished using standard techniques including sequencing, PCR based analytical techniques, hybridization analysis (e.g., Southern or Northern blot analysis) or other known procedures.

In one embodiment a method of treating an NF1-related social learning disorder is provided wherein the method comprises the steps of detecting a defective NF1 gene in a patient and treating the patient having the defective NF1 gene with a pharmaceutical composition comprising an effective amount of a Pak1 inhibitor. In one embodiment the patient is treated by administering a composition comprising at least one Pak1 specific inhibitor.

In one embodiment, a method of treating a social or learning disorder is disclosed. The method comprises the step of administering a pharmaceutical composition comprising at least one Pak1 inhibitor. The dosage of the Pak1 inhibitor in the pharmaceutical composition can vary depending on factors such as the size and age of the patient, the severity of the disorder, and the route of administration of the conjugate. The effective amount to be administered to the patient is based on body surface area, weight, and physician assessment of the condition of the patient. An effective dose can range from about 1 ng/kg to about 1 mg/kg, from about 100 ng/kg to about 500 μg/kg, or from about 100 ng/kg to about 25 μg/kg.

While the invention is susceptible to various modifications and alternative forms, specific embodiments will herein be described in detail. It should be understood, however, that there is no intent to limit the invention to the particular forms described, but on the contrary, the intention is to cover all modifications, equivalents, and alternatives falling within the scope of the invention.

### EXAMPLES

Methods. All experiments were conducted using male mice. Wild-type, Nf1+/−, Nf1+/−/Pak1−/− and Pak1−/− mice bred on a C57BL/6J background were tested at approximately 10 weeks of age. The social interaction test and elevated plus-maze were conducted as previously described (Noll et al. (2007); Shekhar et al. (1993)). Four sessions were conducted including (1) acclimation (two empty cages), (2) preference for social interaction (novel mouse, empty cage), (3) short-term social learning (familiar mouse, novel mouse), and (4) long-term social learning (familiar mouse—24 hr. later, novel mouse). Anxiety-like behavior/avoidance learning was measured using two five minute trials. For the olfactory habituation test, mice were assessed for time spent sniffing cotton tipped swabs. The time spent sniffing cotton swabs scented with either water, almond extract or novel mouse was measured. Following the behavioral tests, mice from each respective strain were used to prepare coronal slices, containing the basolateral amygdala (BLA). Utilizing the whole-cell patch clamp, spontaneous and miniature synaptic currents were recorded. Mouse frontal cortical neurons were aseptically dissected and cultured from each respective genotype. Following dissection, neuronal cells were isolated by dissociation both enzymatically and mechanically, as described previously (Brittain et al. (2009)). Further, cell protein extracts were obtained for Western blot analysis. For the protein expression analysis, the whole cell protein extracts from PFC and BLA regions were obtained. Then, mass spectrometry analysis was performed. Pathway analyses were conducted on proteins that showed significant differences between WT and Nf1+/−, and were restored in the Nf1+/−/Pak1−/−.

### Animals

All experiments were conducted using male mice. The mouse strains tested were bred on a C57BL/6J background and included: (1) Wild-type, (2) Nf1+/−, (3) Nf1+/−/Pak1−/− and (4) Pak1−/− mice. The Nf1+/− mice were obtained from Tyler Jacks at the Massachusetts Institute of Technology (Cambridge, Mass.), and Pak1−/− mice were obtained from Jonathan Chernoff (Fox Chase Cancer Center). To generate the Nf1+/−/Pak1−/− mice, Pak1−/− mice were intercrossed with the Nf1+/− strain. All mice were singly housed, given food and water ad libitum and maintained on a 12 hour light-dark cycle (7:00 am/7:00 pm) at 72° F. The “stimulus” mice used for the social behavior tests were age-matched, adult male C57BL/6J mice that had no previous contact with the “test” mice.

### Social Behavior Tests

The social interaction test was conducted as previously described (Noll, R. B. et al., Am. J. Med. Genet. A. 143A, 2261-2273 (2007)). between 8:00 am and 4:00 pm. Four 10 minute sessions were conducted including (1) acclimation (two empty cages), (2) preference for social interaction (novel mouse, empty cage), (3) short-term social learning (familiar mouse, novel mouse), and (4) long-term social learning (familiar mouse—24 hr. later, novel mouse). Following the “short-term social learning” session, the “novel” mouse was removed from the apparatus, and the “test” mouse was allowed to interact with the “familiar” mouse for an additional 45 minutes. All tests were videotaped and independently scored at a later time by two individuals who were unaware of the animals' genotype.

### Elevated Plus Maze

The elevated plus-maze (EPM) was conducted as previously described (Shekhar et al. (1993)). At the start of the EPM session, the mice were placed in the center chamber of the apparatus with the animal's head facing the open-arm of the EPM and were allowed to freely explore the entire apparatus for five minutes. This procedure was again performed 24 hours later as a measure of avoidance learning as has been previously used to assess PFC-amygdala based aversive learning. An arm entry was defined as having all four paws into the arm of the EPM. Following each test, the apparatus was cleaned with 90% ethanol and then dried.

### Olfactory Habituation Test

The mice were placed in a clean cage, and were assessed for time spent sniffing cotton tipped swabs suspended from the cage lid. The cotton swabs were dipped in (1) water, (2) almond extract (1:100 dilution) or (3) wiped in a zig-zag pattern across the bottom surface of a cage that contained an unfamiliar mouse (a singly housed male mouse (C57BL/6J)). Sequences of three identical swabs were assayed for each odor as follows: water, water, water, almond, almond, almond, unfamiliar cage, unfamiliar cage, unfamiliar cage. Each swab was presented for 2 minutes for a total session lasting 18 min per mouse.

### Isolation And Culture of Neuronal Cells From Murine Strains

Mouse frontal cortical neurons were aseptically dissected and cultured from each respective genotype (WT, Nf1+/−, Nf1+/−/Pak1−/−, Pak1−/−). Following dissection of each respective brain region, neuronal cells were isolated by dissociation both enzymatically and mechanically (via trituration through a flame-polished Pasteur pipette) in a Papain solution (12 units/ml; Worthington) as described previously (Brittain et al. J. Biol. Chem. 284, 31375-31390 (2009)). For this experiment, the neuronal cultures were assigned to one of two experimental conditions: (i) at basal levels and (ii) following the application of recombinant murine stem cell factor (rmSCF; PreproTech) at 10 ng/ml. rmSCF was applied to the neuronal cultures for 2 minutes. The cells were then washed with ice-cold PBS and lysed in buffer, as described below.

### Immunoblotting ERK/p-ERK In Mouse Cortical Neurons

Whole cell protein extracts were obtained from cultured frontal cortical neurons in lysis buffer (50 mM Tris pH 7.4, 150 mM NaCl, 2 mM EDTA pH 8.0, 1% Triton X-100, 1 mM PMSF, 1 mM NaF, 1 mM Na3VO4, 10% glycerol and protease inhibitors). The samples were sonicated and cellular debris was removed by centrifugation at 13,000 g for 30 min at 4° C. Protein concentrations were determined using a BCA assay (Thermo Scientific). Equivalent amounts of protein was electrophoresed on 10% SDS-PAGE gels, transferred to PVDF membranes (GE Healthcare, Little Chalfont, UK), and detected by Western blotting using the ECL Plus system (Amersham Biosciences). Antibodies used were Phospho-ERK Antibody (Cell Signaling Technology), ERK1 Antibody (Cell Signaling Technology), and GAPDH (Millipore).

### Electrophysiology

The electrophysiological methods were performed as described previously (Molosh et al. Neuropsychopharmacology 35, 1333-1347 (2010)). Briefly, following decapitation, the brains were rapidly removed and placed in oxygenated artificial cerebrospinal fluid (ACSF) [130 mM NaCl; 3.5 mM KCl; 1.1 mM KH2PO4; 1.3 mM MgCl2; 2.5 mM CaCl2; 30 mM NaHCO3; 10 mM glucose], and coronal slices (350 μM) were prepared containing the basolateral amygdala (BLA). Prior to recording, slices were incubated at room temperature for 1 hr. in oxygenated ACSF [95% O2/5% CO2 mixture]. Slices were then transferred to a submersion-type slice chamber mounted on the stage of a Nikon E600FN Eclipse (Nikon Instruments, Melville, N.Y.) microscope and perfused with ACSF [1-2 ml per minute] heated to 30° C. Whole-cell patch-clamp recordings were obtained using standard techniques. Borosilicate glass electrodes (WPI, Sarasota, Fla.) (resistance 3-6 MΩ) were prepared with a potassium gluconate based recording solution [130 mM K-Gluconate, 3 mM KCl, 3 mM MgCl2, 5 mM phosphocreatine, 2 mM K-ATP, 0.2 mM NaGTP, 10 mM HEPES] and were maintained at a holding potential of −60 mV. Whole-cell access resistances were monitored throughout each experiment and ranged from 5-20 MΩ; a change of 15% was deemed acceptable. Projection neurons were identified according to their characteristic size and shape. At the start of each experiment a series of standardized current clamp protocols were performed to further validate the identity of BLA projection neurons. Drugs were then applied by adding them directly into the ACSF at the required concentration. The sIPSC were recorded at a holding potential of −55 mV in the presence of DNQX (20 μM), (RS)CPP (10 μM). The sEPSC were acquired at a holding potential of −60 mV in the presence of bicuculline methochloride (10 μM) and CGP 52432 (1 μM). The sIPSC and miniature IPSC (mIPSC) [in the presence of 1 μM tetrodotoxin (TTX)] were captured continuously for 1 min at a sampling frequency of 20 kHz. Spontaneous currents were detected and analyzed using the pClamp 10.2 (Molecular Devices, Sunnyvale, Calif.). All chemicals, except (RS)CPP and CGP 52432 (Tocris Biosciences, Ellisville, Mo.), were purchased from Sigma-Aldrich (St. Louis, Mo.).

### Statistical Analyses of Behavioral And Electrophysiological Data

For behavior tests, data were analyzed using one-way or two-way analyses of variance (ANOVAs) or repeated measures ANOVAs. Sociability and social novelty preference were evaluated using within-genotype repeated measures ANOVAs, using cage occupancy (e.g., novel mouse or “familiar” mouse) as the factor. Significant effects were further analyzed using post hoc Fisher's protected least significant difference (PLSD) tests. For electrophysiology, statistical analyses included paired t-test and one-way ANOVAs using a Dunnet's post-hoc. For all comparisons the confidence level for significance was set at P<0.05.

### Protein Expression Analysis

Following decapitation tissue slices were homogenized in 50 mM ammonium bicarbonate, and whole cell protein extracts were obtained from brain slices in lysis buffer (30 mM Tris, pH 7.4, 150 mM NaCl, 1% Triton X-100, 0.1% SDS, 1 mM PMSF, 10 mM EDTA, 1 mM Na2CO3, 160 mM NaF, complete protease inhibitor) with ProteoSpin total protein detergent clean up micro kit (Norgen, Canada). BCA Protein Assay Kit (Pierce, Rockford, Ill.) was utilized to determine protein concentrations of the lysates (Pierce, Rockford, Ill.). Protein samples were reduced through DTT and alkylation was achieved by adding IAA. The protein samples then subjected to tryptic digestion at 37° C. overnight and quenched through the addition of neat formic acid. LC-MS/MS analyses of the tryptic digests were performed using a Dionex 3000 Ultimate nano-LC system (Dionex, Sunnyvale, Calif.) interfaced to LTQ Orbitrap hybrid mass spectrometer (Thermo Scientific, San Jose, Calif.). Prior to separation, a 2-μl aliquot of trypsin digestion (1.5 μg protein equivalent) was loaded on PepMap300 C18 cartridge (5 μm, 300 Å, Dionex) and eluted through the analytical column (150 mm×100 μm i.d, 200 Å pores) packed with C18 magic (Michrom Bioresources, Auburn, Calif.). Peptides originating from protein tryptic digests were separated using a reversed-phase gradient from 3-55% B, 99.9% acetonitrile with 0.1% formic at 500 nl/min flow rate and passed through an ADVANCE ionization source (Michrom Bioresources, Auburn, Calif.). Switching between MS scan and CID-MS, eluted LC products undergo an initial full-spectrum MS scan from m/z 300 to 2000 in the Orbitrap at 15,000 mass resolutions. Subsequently CID-MS (at 35% normalized collision energy) was performed. The total cycle (6 scans) is continuously repeated for the entire LC-MS run under data-dependent conditions. Mascot version 2.1.3 was used for all search results against Swiss-Prot database for house mouse, and the quantitative analysis of proteins was carried out using ProteinQuant Suit developed at Indiana University (Mann, B. et al. Rapid Commun. Mass Spectrom. 22, 3823-3834 (2008). The combined master files were then incorporated with their corresponding mzXML files were submitted to ProteinQuant as described previously (Rapid Commun. Mass Spectrom. 22, 3823-3834 (2008)).

Pathway analyses were then conducted on proteins that showed significant differences between WT and Nf1+/−, and were restored in the Nf1+/−/Pak1−/− genotype utilizing INGENUITY systems software (Redwood City, Calif.). Protein expression data were further analyzed using one-way and two-way analyses of variance (ANOVAs: see table 1). In the presence of significance (P<0.05) with either ANOVA, post hoc Fisher's protected least significant difference (PLSD) tests were done to detect specific differences between groups. Only proteins that showed significant differences between WT and Nf1+/−, and were restored to WT levels in the Nf1+/−/Pak1−/− genotype are listed.

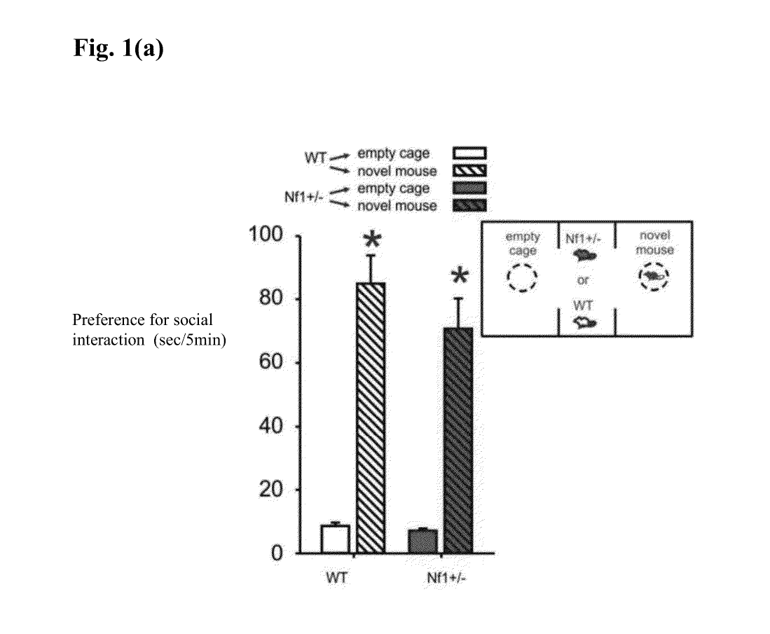

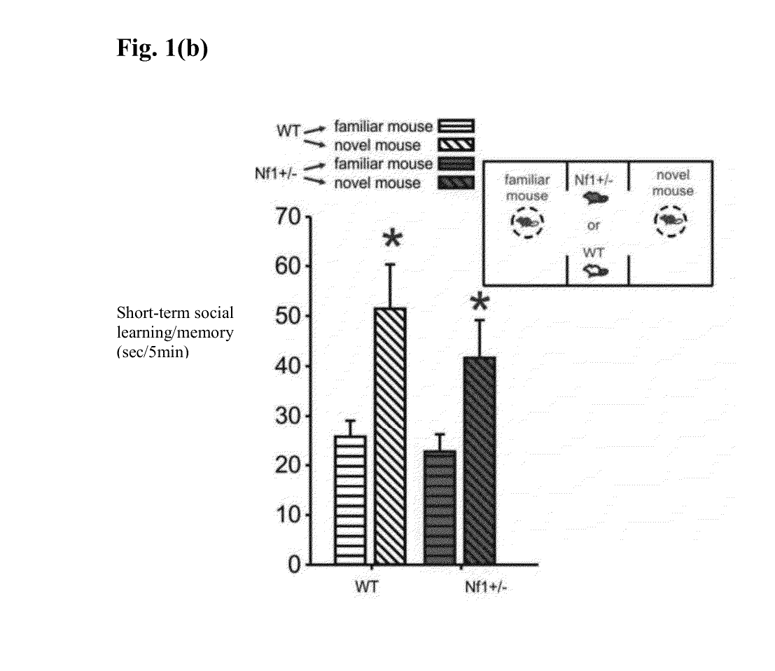

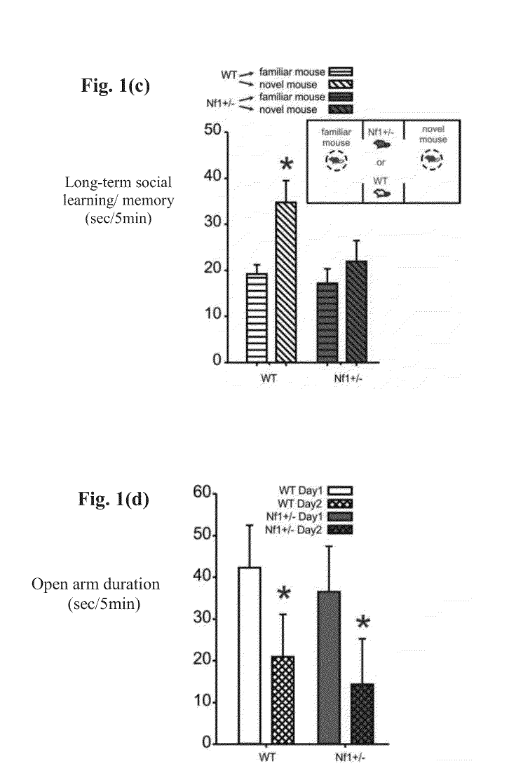

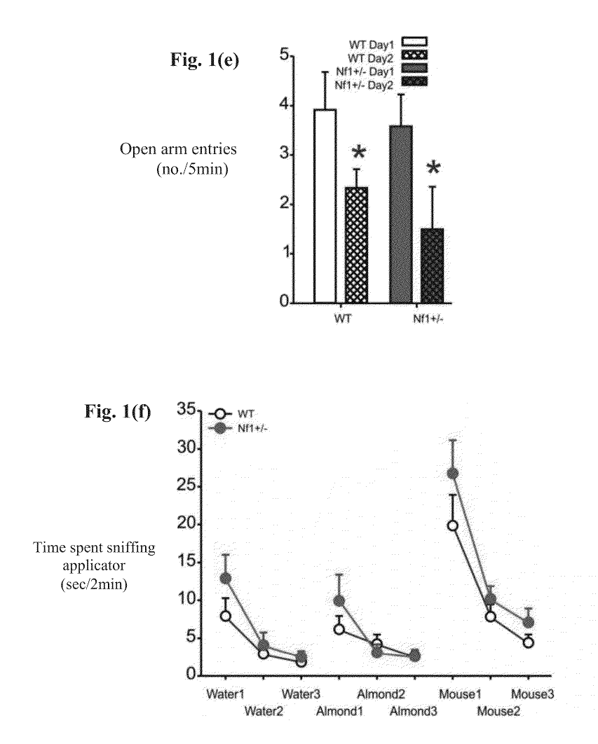

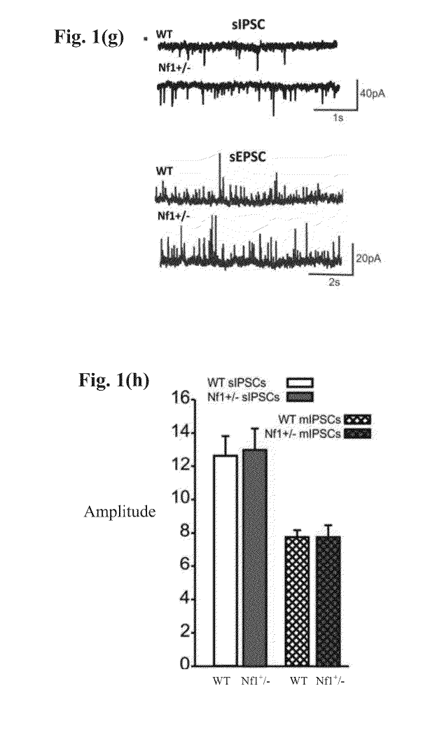

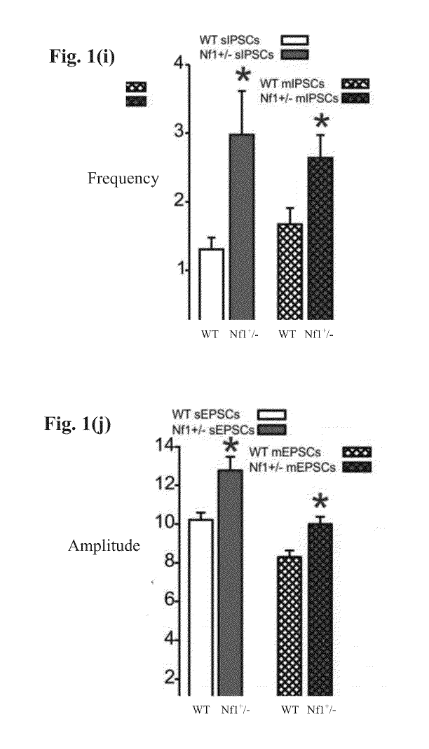

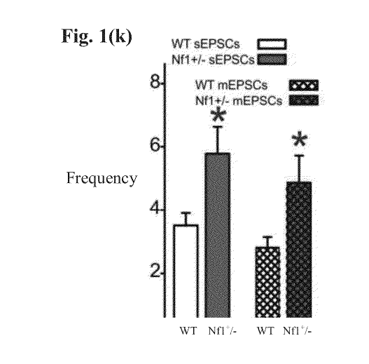

Social learning test. To determine whether the heterozygous mutation of the Nf1+/− in male mice affects social learning, a three-chambered apparatus with wire cages placed at opposite ends of the apparatus to house “stimulus” mice was used (Sankoorikal et al. (2006)). Both WT and Nf1+/− strains spent significantly more time sniffing the cage with a novel mouse over the empty cage [F1, 22=105.8, p<0.001 for novel verses empty cage; FIG. 1a], demonstrating that the Nf1+/− genotype does not affect general social cue recognition in mice (baseline social behavior). When presented with a choice between a novel mouse and a familiar mouse, mice spend more time interacting with a novel mouse and this behavior is utilized to test the ability of mice to discriminate social cues. Both WT and Nf1+/− genotypes spent significantly more time exploring the cage with the novel mouse [F1, 22=15.1, p=0.001 for novel verses familiar mouse; FIG. 1b], suggesting that Nf1+/− mice show intact ability to discriminate social cues. However, when presented with a novel mouse and the same “familiar” mouse 24 hours later, the Nf1+/− mice fail to recognize the “familiar” mouse from the previous day, whereas the WT mice demonstrate robust long-term social learning [F1, 22=11.7, p=0.002 for novel mouse verses familiar mouse; FIG. 1c]. These results indicate that Nf1+/− mice while show normal social preference at baseline, are unable to retain or recall the social memory and discriminate between familiar and unfamiliar social cues following a 24 hour delay.

Avoidance of previously known aversive condition. The Nf1+/− mice were also tested in another behavioral learning paradigm that employs amygdala-cortical circuits, namely avoidance of previously known aversive condition. Retention of memory for an aversive cue and the ability to demonstrate avoidance of fear stimulus 24 hours later were tested by utilizing the elevated plus-maze. When tested 24 hours later, both genotypes showed similar increases in the avoidance of the open arms, suggesting normal retention of memory for aversive cues and avoidance in both genotypes (FIG. 1d-e). Differences in the ability to discriminate olfactory cues is another possible confound that could result in mice showing poor social learning.

Electrophysiology of basolateral amygdala neurons. Functional changes in the amygdala networks in Nf1+/− mice were characterized using whole-cell patch-clamp from BLA projection neurons to study spontaneous excitatory and inhibitory synaptic currents (sEPSC and sIPSC, respectively). sEPSCs were characterized in the presence of GABAA and GABAB antagonists (1 μM CGP 52432 and 10 μM bicuculline methochloride), whereas sIPSCs were studied in the presence of AMPA and NMDA antagonists (20 μM DNQX and 10 μM CPP). Additionally, miniature EPSCs (mEPSC) and IPSCs (mIPSC) were recorded in the presence of 1 μM tetrodotoxin (TTX). While projection neurons of the BLA from Nf1+/− mice exhibited no differences in either sIPSC or mIPSC amplitudes, they show significant increases in both sIPSC and mIPSC frequency as compared to WT [(t10=2.518, p=0.02) for Nf1+/− versus WT (t10=2.368, p=0.028) respectively, n=11; FIG. 1g-h]. These changes suggest increases in presynaptic GABA release in the BLA. In contrast to the IPSCs, both sEPSCs and mEPSCs from projections neurons in the BLA of Nf1+/− mice showed significant increases in amplitudes, (sEPSC: t30=3.339 p=0.002; mEPSC: t30=3.394, p=0.002) and frequency (sEPSC: t30=2.547, p=0.016; mEPSC: t30=2.364, p=0.025). These findings suggest both pre- and post-synaptic changes in glutamate neurotransmission [FIG. 1i, j].

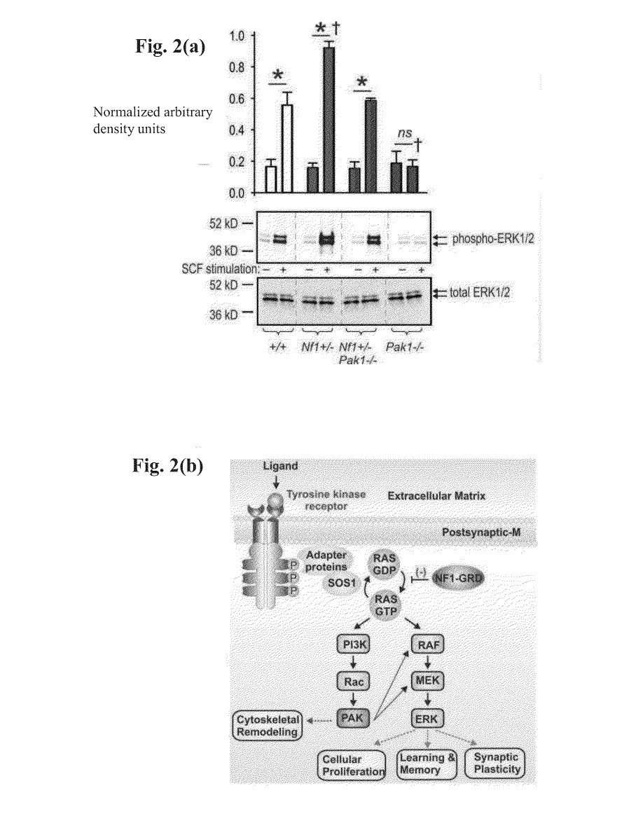

Activation of MAPK. Neurons cultured from Nf1+/− mice were tested to see if they would display a similar hyperactivation of MAPK following growth factor stimulation, and whether this abnormality would be rescued by the co-deletion of Pak1. Western blotting was used to quantify phosphorylated ERK1/2 (p-ERK1/2) in cultured cortical neurons at basal levels and 2 minutes following application of recombinant murine Stem Cell Factor (rmSCF; 10 ng/ml). SCF was chosen as the growth factor since this was previously used to demonstrate cellular effects of NF1 deletion in peripheral cells and there is emerging information that SCF receptors are present on cortical neurons and are involved in hippocampal memory formation. Application of SCF increased the p-ERK1/2 levels to significantly greater degree in neurons cultured from Nf1+/− mice when compared to those cultured from WT mice, and this p-ERK1/2 hyperactivation was normalized to levels consistent with WT in cortical neurons cultured from Nf1+/−/Pak1−/− mice (FIG. 2a). Thus, Pak1 co-deletion appears to normalize the MAPK hyperactivity induced by Nf1 deletion in the central nervous system similar to the results seen in mast cells in peripheral regions.

### EXAMPLE 2

### Nf1+/− And Pak1−/− Double Knockout Animals

### Behavioral Tests

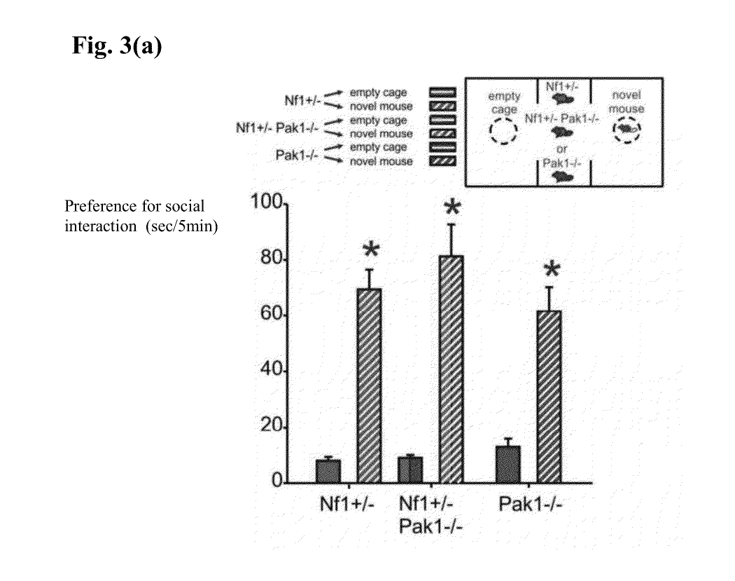

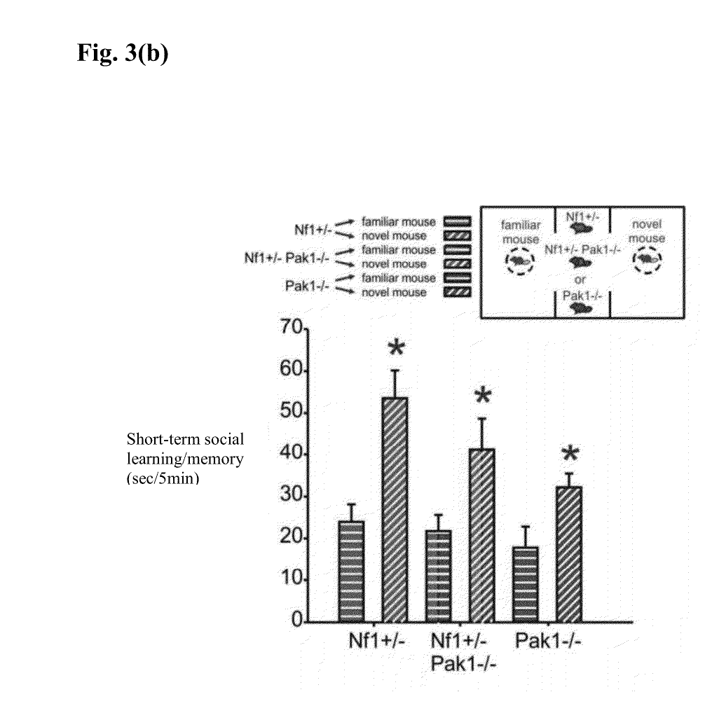

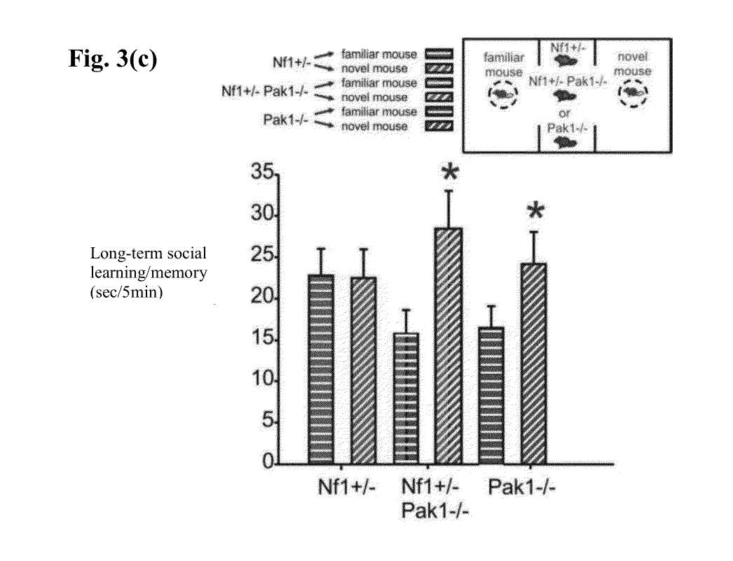

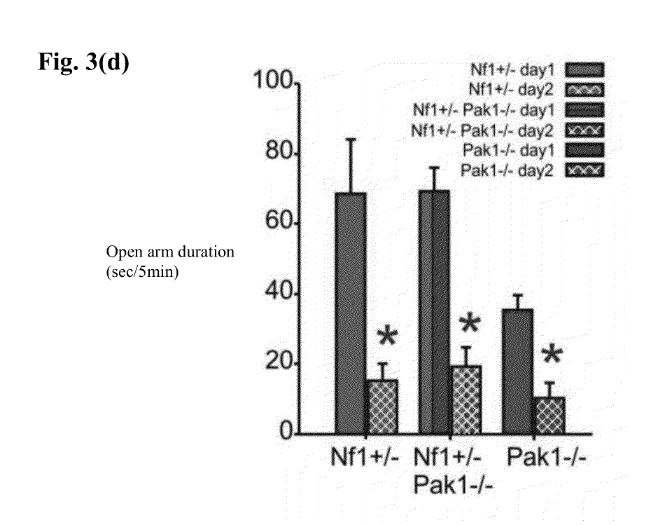

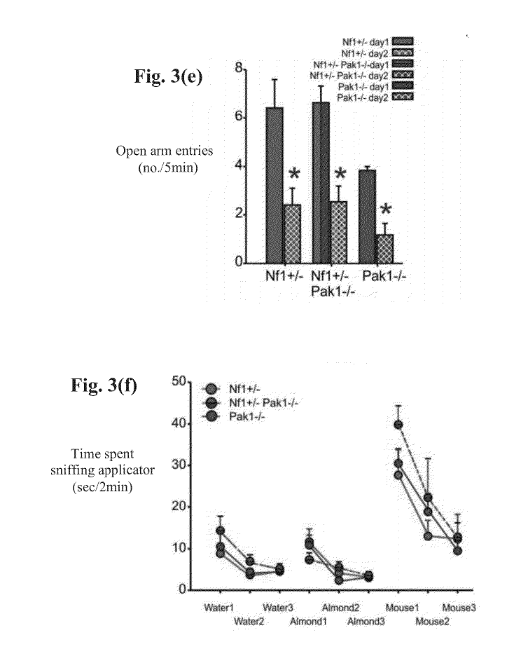

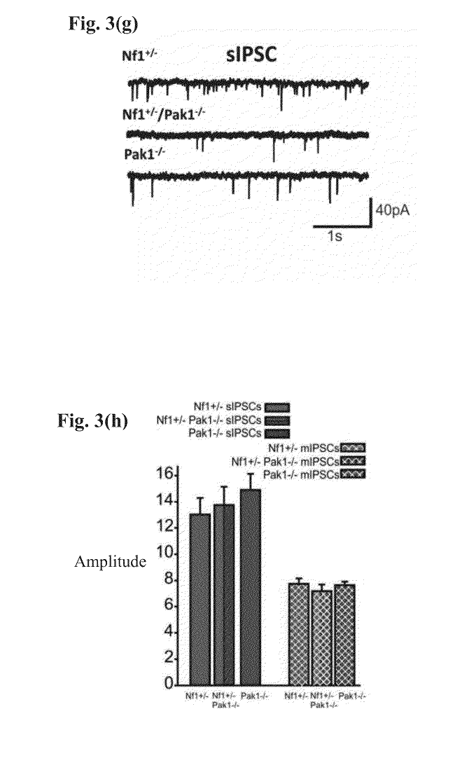

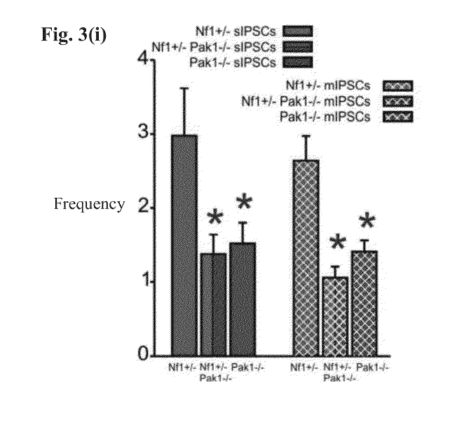

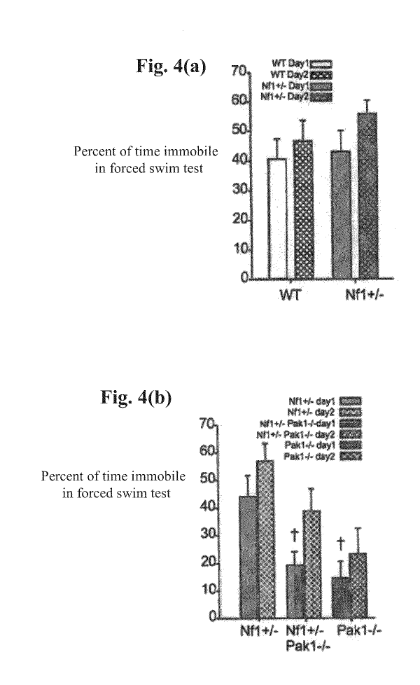

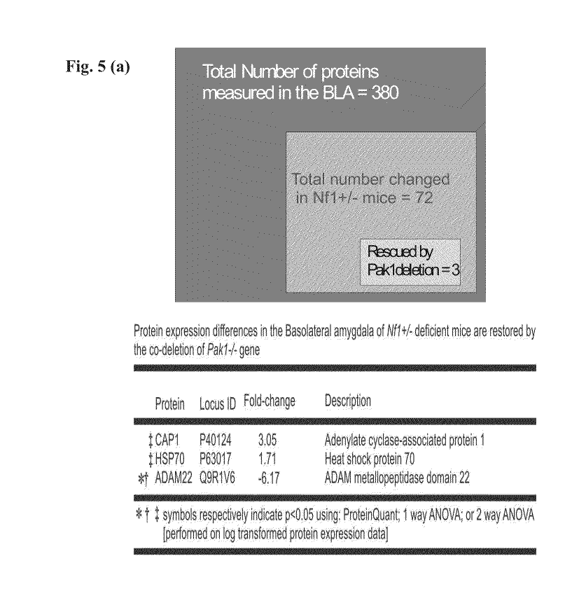

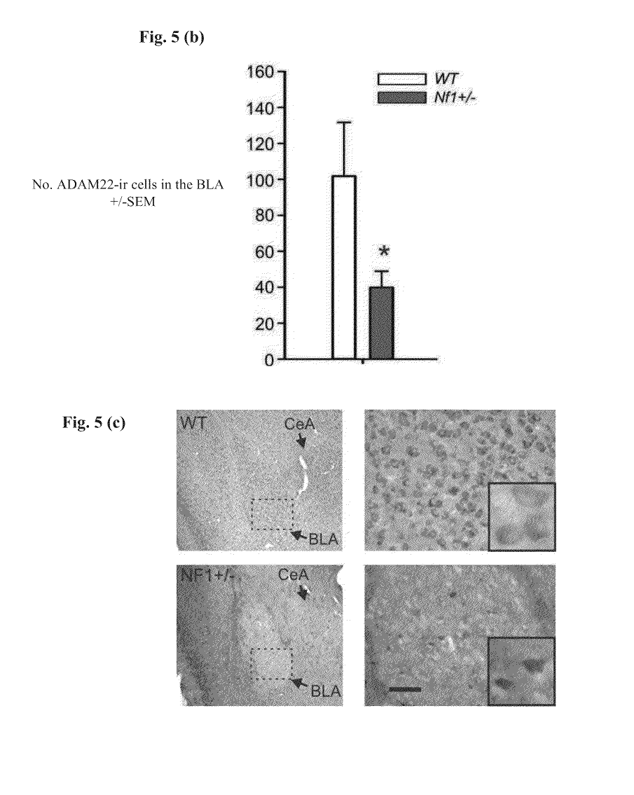

The genetic intercross (Nf1+/−/Pak1−/−) was tested to see if it would restore the learning deficits seen in Nf1+/− mice. We found that Nf1+/−, Nf1+/−/Pak1−/−, and Pak1−/− strains show no differences in their preference for social interaction, [F1, 26=100.5, p=0.001 for novel verses empty cage; FIG. 3a] or short-term social learning [F1, 26=32.6, p=0.001 for novel verses familiar mouse; FIG. 3b]. However, as before, deficits were seen in Nf1+/− mice in long-term social learning [F1, 26=7.3, p=0.01; FIG. 3c], but importantly, co-deletion of Pak1 in Nf1+/− mice (Nf1+/−/Pak1−/− mice) clearly restored this learning deficit. Similar to Applicants' previous results with Nf1+/− mice, no differences in anxiety, avoidance learning or olfaction in the Nf1+/−/Pak1−/− mice were detected (FIG. 3d-f). Consistent with the biochemical and behavioral effects, co-deletion of Pak1 completely normalized both the increases in sIPSC and mIPSC frequencies [F2, 26=4.606, p=0.016 for Nf1+/−/Pak1−/− and Pak1−/− versus Nf1+/−, F2, 26=14.54, p<0.0001, respectively; FIG. 3d-f], confirming that Pak1 deletion also restores the disruption in the BLA network induced by Nf1 mutation.

### Protein Expression

Differences in protein expression were screened in two key areas implicated in social learning, namely the BLA and PFC. Utilizing mass spectrometry, protein expression levels were measured in these two regions in WT, Nf1+/−, Nf1+/−/Pak1−/− and Pak1−/− mice. Specific proteins were identified that were changed in Nf1+/−, compared to WT, and those that were restored in Nf1+/−/Pak1−/− mice but were not similarly altered by deletion of Pak1 gene alone. INGENUITY systems software (Redwood City, Calif.) was then used to conduct pathway analyses to identify protein networks that were disrupted in Nf1+/− mice and returned to WT levels in Nf1+/−/Pak1−/− mice. Based on Applicants' selection algorithm, a series of proteins implicated in glutamate and GABA neurotransmission as well as synaptic plasticity were disrupted in Nf1+/−-mice, all of which were comparable to WT levels in Nf1+/−/Pak1−/− mice (Table. 1). A number of these proteins such as LSAMP, EAA1, drebrin, dynamin 1 and ADAM 22 have already been specifically implicated in regulation of neurotransmission, synaptic plasticity as well as learning and social behaviors. Thus, Nf1 and Pak1 genes also have opposing effects on the expression of proteins important for synaptic plasticity and learning in the two key brain areas implicated in social learning.

TABLE 1
Representation of protein expression differences seen in Nf1+/−
deficient mice that are rescued by the co-deletion of Pak1−/− gene.
Columns represent proteins from the prefrontal cortex and basolateral
amygdala showing full or partial rescue with Pak1−/− codeletion
with Nf1+/−. Column 2 is the locus ID of each protein. Column 3
represents fold changes in protein expression in Nf1+/− mice
compared to Wt controls. Column 4 represents definitions for
abbreviated proteins in column 1. Protein expression differences
in the PFC and BLA of Nf1 deficient mice are restored by the
co-deletion of Pak1 gene

| | | | |
|---|---|---|---|
| Protein | Locus ID | Fold-change | Description |

Prefrontal cortex

| | | | |
|---|---|---|---|
| *†‡LSAMP | Q8BLK3 | 8.25 | Limbic system-associated |
| | | | membrane protein |
| †RLA1 | P47955 | 7.95 | Acidic ribosomal subunit |
| | | | P1 |
| *†‡CALB1 | P12658 | 6.21 | Calbindin |
| *†‡ACTS | P68134 | 5.15 | Alpha-actin-1 |
| *†HNRH1 | O35737 | 4.07 | Heterogeneous nuclear |
| | | | ribonucleoprotein H |
| *†‡CAPR1 | Q60865 | 3.53 | Caprin-1 |
| *†H2A2B | Q64522 | 3.44 | Histone H2A type 2-B |
| ‡EAA1 | P56564 | 3.41 | Excitatory amino acid |
| | | | tranporter 1 subtype |
| *†NUCL | P09405 | 3.21 | Nucleolin |
| *†‡AP1B1 | O35643 | 2.87 | AP-1 complex subunit |
| | | | beta-1 |
| ‡PP2AA | P63330 | 2.87 | Protein phosphatase |
| | | | 2A alpha subunit |
| *†‡MBP | P04379 | 2.47 | Myelin basic protein |
| *†DREB | Q9QXS6 | 2.03 | Drebrin |
| *†CLCB | Q6IRU5 | 1.86 | Clathrin light Chain B |
| *†‡THY1 | P01831 | 1.57 | Thy-1 membrane |
| | | | glycoprotein |
| *†‡MAP1A | Q9QYR6 | 1.49 | Micotubule-associated |
| | | | protein 1A |
| *†‡ENOA | P17182 | −1.37 | Alpha-enolase |
| *†‡STXB1 | O08599 | −1.63 | Syntaxin-binding protein 1 |
| *†DHE | P26443 | −1.88 | Glutamate dehydrogenase 1 |
| *†‡LDHA | P06151 | −2.02 | L-lactate dehydrogenase A chain |
| *†DYL2 | Q9D0M5 | −2.27 | Dynein light chain 2 |
| *†DYN1 | P39053 | −2.56 | Dynamin-1 |
| *†NACAM | P70670 | −3.12 | Nascent-associated |
| | | | Complex subunit alpha |
| *†‡CAZA2 | P47754 | −3.57 | F-actin-capping protein |
| | | | subunit alpha-2 |

Basolateral amygdala

| | | | |
|---|---|---|---|
| ‡CAP1 | P40124 | 3.05 | Adenylate cyclase- |
| | | | associated protein 1 |
| ‡HSP70 | P63017 | 1.71 | Heat shock protein 70 |
| *†ADAM22 | Q9R1V6 | −6.17 | ADAM metallopeptidase |
| | | | domain 22 |

*†‡symbols respectively indicate p < 0.05 using: ProteinQuant; 1 way ANOVA; or 2 way ANOVA [performed on log transformed protein expression data]

In conclusion, our findings demonstrate that Nf1 mice demonstrate a selective deficit in ‘social learning’ that is rescued by co-deletion of Pak1 gene. This is quite unlike the recently reported Shank3 mutant mice which prefer interacting with the empty cage and show deficits in overall social behaviors at baseline26. The mechanisms underlying the opposing regulation of social learning by these genes appears to involve disruption of GABA-mediated inhibition and glutamate excitation of the projection neurons of the amygdala and altered expression of important synaptic proteins in the amygdala and PFC. Our results also suggest that Pak1 inhibitors may represent a therapeutic target of interest for the treatment of Nf1-related social and learning disorders.

### EXAMPLE 3

Administration of Pak1 inhibitors to NF1 mutant mice ameliorates deficits in social learning test

The two major pathways by which Nf1 deletion could cause the behavioral effects include MAPK or Pak1 activation. Pharmacologically blocking one of these pathways in Nf1+/− mice and demonstrating if it rescues the social learning deficits would be an important to elucidate mechanisms. Therefore, we will pre-treat Wt and Nf1+/− mice with drugs that block one of the pathways and test them in the social learning tasks.

Experimental Methods: For testing the involvement of each of the above mentioned pathways, 24 Wt and Nf1+/− mice per drug will be divided into 3 groups of 8, and assigned to receive vehicle or one of the 2 doses of the following drugs intraperitoneally/icv 30 min before day 1 of the social interaction/learning task.

Drugs and Doses Employed to block signaling pathways: The following inhibitor drugs will be utilized to block the select signaling pathway. SL327 (α-[amino[(4-aminophenyl)thio]methylene]-2-(trifluoromethyl) benzeneacetonitrile) (100 mg/kg), a selective inhibitor of the upstream ERK activator MEK, and PD98059 will be used to block the MAPK pathways (Papadeas, 2008). In order to block the Pak1 pathways, we will use the small molecule IPA-3 (1) (Deacon et al., 2008). The doses are based on published studies and we will inject them via the intraperitoneal or intracerebroventiricular (i.c.v) route in accordance with the schedule indicated in Table 2.

TABLE 2

| Path- | | | | |
|---|---|---|---|---|
| way | Drug | Dose | Source | Reference |
| Pak1 | IPA-3 | 10 & 40 ug/100 ul | Peptide Core | Deacon et al., |
| | | i.c.v. | | 2008 |
| MAPK | PD98059 | 5 and 25 uM/100 ul | BMS | Papadeas et al., |
| | | i.c.v | | 2008 |
| | SL327 | 30 & 100 mg/kg | Calbiochem | Papadeas et al., |
| | | i.p. | | 2008 |

We anticipate that Nf1+/− mice that are treated with vehicle will exhibit social memory deficits as before. These behavioral changes will be at least partially attenuated in mice that receive i.p. injections of IPA-3, PD98059 and/or 51327. We would expect these responses to be associated with pharmacological selectivity to MAPK and reduction of pERK levels.

### General Methods

Animals The mouse strains are bred on C 57BL/6J background and include:

(1) Wild-type (Wt);

(2) Nf1+/−; and

(3) Nf1+/−; Pak1−/− mice.

The Nf1+/− mice are obtained from Tyler Jacks at the Massachusetts Institute of Technology (Cambridge, Mass.), and Pak1−/− mice are obtained from Dr. Jonathan Chernoff (Fox Chase Cancer Center). To generate the Nf1+/−; Pak1−/− mice, Pak1−/− mice are intercrossed with the Nf1+/− strain and then genotyped as previously described (McDaniel et al, 2008). The “stimulus” mice for the social behavior tests are age-matched, adult male C57BL/6J mice without previous contact with the “test” mice.

Behavioral Protocol: For behavioral testing, the mice are acclimated to the animal facility for 6 days and are habituated to the testing room for 30 minutes before testing began. The housing facility is maintained on a 12 hour light-dark cycle (7:00 am/7:00 pm) at 72° F. All tests are videotaped and independently scored by two individuals who are unaware of the genotype. A modified version of the social interaction test is conducted as previously described by Sankoorikal et al, (2006). Briefly, the testing apparatus consisted of a rectangular box (20 inch length×10 inch width×9 inch height) with three interconnecting chambers. The two chambers at each end of the apparatus are equal in size (7.5 inch×10 inch), while the middle chamber is slightly smaller (4.75 inch×10 inch). In addition, two identical cylinders (3 inch diameter, 5 inch height) constructed of wire are placed at each end chamber. The diameter and height of the cylinders are sufficient for the “stimulus” mouse to move comfortably, and the openings in the wire cage are evenly spaced and allowed for auditory, visual, and olfactory investigation as well as some tactile contact. Four 10 minute sessions are conducted including (1) acclimation (two empty cages), (2) social interaction (novel mouse, empty cage), (3) social preference (familiar mouse, novel mouse), and (4) social learning (familiar mouse, novel mouse). Other than the social learning test which is performed on the following day, the test sessions are conducted in sequential order directly following the previous session. Following each session, the apparatus is cleaned with 90% ethanol and then dried, and the flooring is replaced with a clean mat.

Surgical Techniques: Implantation of chronic microinjection cannulae: Unilateral chronic microinjection cannulae are stereotaxically guided to the lateral cerebral ventricle site using stereotaxic coordinates for the mouse (Paxinos and Watson, 1992). Microinjection: Acute microinjections of drugs are done with injection cannulae (33 gauge, Plastics One Products, Roanoke, Va.) connected to a Hamilton 1 μl syringe and Sage pumps. Most of the studies will be done with an injection volume of 100-250 nl per side as needed.

Data analysis: A one-way repeated measure ANOVA will be used to analyze the majority of data. When p<0.05 a Tukey's post hoc test will also utilized to make individual comparisons between groups. A student's t-test will also be used for data analysis when only two conditions are being compared. For electrophysiology, the minimum number of cells to be recorded will be 8 per group.

Utilizing mice with a deletion of a single Nf1 gene (Nf1+/−), demonstrated herein is a selective social learning deficit and disruptions in GABA-mediated inhibition and glutamate-mediated excitation of projection neurons of the amygdala, a key structure implicated in social behaviors. It is further demonstrated that neurons from Nf1+/− mice show greater activation of MAPK pathway following growth factor stimulation, and this abnormality can be rescued by the co-deletion of p21-activated kinase 1 (Pak1) gene. Utilizing Nf1+/−/Pak1−/− mice, it is shown herein that the co-deletion of Pak1 restores both disruption of social learning and disrupted inhibition of amygdala neurons seen in Nf1+/− mice. Furthermore, proteomics analyses of brain tissue from Nf1+/− mice identified reduction in key proteins associated with glutamate neurotransmission and synaptic plasticity in the frontal cortex and the amygdala, all of which were normalized in Nf1+/−/Pak1−/− mice. Together, these findings show that administration of a Pak1 inhibitor is useful for treating underlying social learning deficits in NF1 patients and some genetic forms of ASDs.

While the invention has been illustrated and described in detail in the foregoing description, such an illustration and description is to be considered as exemplary and not restrictive in character, it being understood that only the illustrative embodiments have been described and that all changes and modifications that come within the scope of the invention are desired to be protected. Those of ordinary skill in the art may readily devise their own implementations that incorporate one or more of the features described herein, and thus fall within the scope of the present invention.

What is claimed is:

1. A method for treating a social learning disability in a patient, the method comprising the step of
identifying said patient with a social learning disability;
administering to said patient a pharmaceutical composition comprising an effective amount of at least one Pak1 inhibitor.

2. The method of claim 1 wherein the step of identifying said patient with a social learning disability comprises the step of screening patients for a defect in NF1 activity.

3. The method of claim 2 wherein said patient has a modification to the NF1 gene that decreases or eliminates expression of the NF1 gene.

4. The method of claim 2 wherein the Pak1 inhibitor is selected from the group consisting of staurosporine, 3-hydroxystaurosporine, K252a, CEP-1347, OSU-03012, DW12, FL172, IPA3, PF-3758309, PAK10, EKB569, TKI258, and SU-14813.

5. The method of claim 4 wherein the Pak1 inhibitor is a Pak1 specific inhibitor.

6. The method of claim 1 wherein the social learning disability is exhibited as a behavior selected form the group consisting of an inability to read the social cues of others, an inability to modulate situationally inappropriate behavior and an inability to respond appropriately to facial expressions or body language of others.

7. The method of claim 1 wherein the patient has an attention deficit hyperactivity disorder.

8. A method of treating a patient with social learning disability and defective NF1 activity, said method comprising the steps of
screening patients identified as having a social learning disability to identify a subset of patients that also have defective NF1 activity;
administering to said subset of patients a pharmaceutical composition comprising an effective amount of a p21-activated kinase inhibitor.

9. The method of claim 8 wherein the pharmaceutical composition comprises a Pak1 specific inhibitor.

10. The method of claim 8 wherein the defective NF1 activity results from a mutation in an NF1 gene.
